(19) United States
(12) Patent Application Publication
(10) Pub. No.: US 2009/0247564 A1
(43) Pub. Date: Oct. 1, 2009

(54) Crystalline Form of Vinflunine Ditartrate

(76) Inventors: Jean-Louis Maurel, Burlats (FR); Richard Pena, Vielmur Sur Agout (FR); Jean-Paul Ribet, Mazamet (FR)

Correspondence Address: BIRCH STEWART KOLASCH & BIRCH, PO BOX 747, FALLS CHURCH, VA, 22040-0747, US

(21) Appl. No.: 12/086,773

(22) PCT Filed: Dec. 18, 2006
(86) PCT No.: PCT/EP2006/069843

§ 371 Date: Jun. 19, 2008

Related U.S. Application Data
(60) Provisional application No. 60/774,201, filed on Feb. 17, 2006.

(30) Foreign Application Priority Data
Dec. 20, 2005 (FR) 0512942

(51) Int. Cl. A61K 31/4375; C07D 487/08

(52) U.S. Cl. 514/283; 540/477

(57) ABSTRACT

The present invention relates to a novel crystalline form of vinflunine, to a process for preparing it, and to its uses in the therapeutic field, in particular for treating cancer.

The present invention relates to a novel crystalline form of vinflunine, to the process for obtaining it and to its uses in the therapeutic field.

Vinflunine is an indole derivative of the vinblastine and vincristine family.

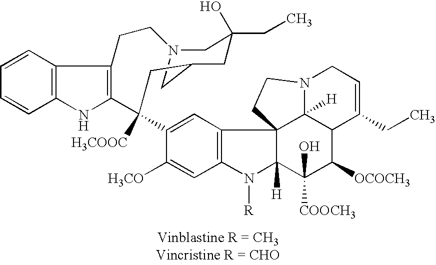

These compounds form part of the antimitotic alkaloids, extracted from Catharanthus roseus, and have been used for many years in anticancer chemotherapy. The difficulties in obtaining these derivatives by extraction from plants have led several research groups to identify similar novel substances having the same properties and to develop a process for obtaining them via semisynthesis. Thus, yindesine and vinorelbine (Navelbine) have been obtained and marketed for cancer treatment. The chemical structure of these compounds has as main feature a combination of two alkaloids monomers, catharanthine and vindoline.

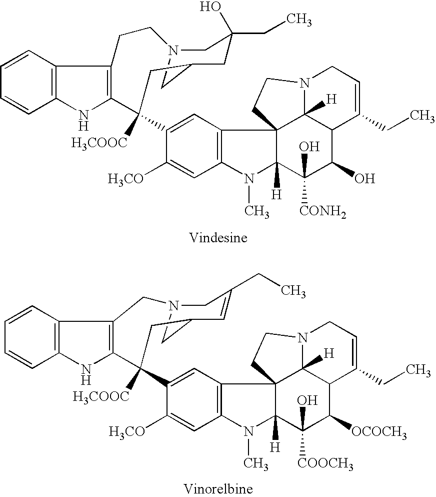

In the context of developing novel synthetic routes for obtaining vinorelbine, the reactivity of this compound in superacid medium has led to the identification of a novel molecule, 20′,20′-difluoro:3′,4′-dihydrovinorelbine, or vinflunine (WO95/03312). The therapeutic value of this compound was also checked in the course of these same studies.

The exact conformation of vinflunine was studied by various methods of ¹H NMR and ¹³C NMR spectroscopy (Magn. Reson. Chem., 2001, 39, pp. 43-48). This study was conducted on vinflunine ditartrate in solution. However, this salt has hygroscopic properties that limit its stability in solid form, and are a handicap during industrial manufacture. To date, it has been isolated only in the form of an amorphous powdery solid that has to be stored at a negative temperature, below −15° C., and under an inert gas atmosphere, for example under nitrogen or argon. This compound is difficult to handle and store, and any form that improves the physical stability in the solid state would simplify the manufacturing, storage and packaging processes.

Conventionally, the crystallization of an amorphous compound can present major difficulties, and obtaining the first crystals is always problematic. However, this type of solid form allows a large number of drawbacks of the amorphous form to be overcome. Specifically, it retains less water, and its improved stability over time facilitates its handling during industrial manufacturing processes by virtue especially of a reduced tendency to aggregate as a cake, and better flowability. It also allows more varied galenical forms to be envisaged, and to facilitate its manufacturing and handling.

The Applicant has demonstrated that it is possible to crystallize vinflunine ditartrate, by using a suitable solvent system.

Thus, one subject of the present invention is crystalline vinflunine ditartrate of formula (I):

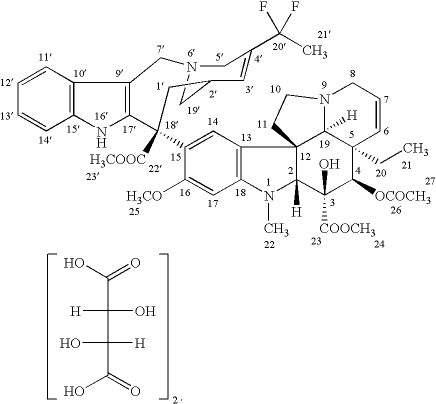

Preferably, the invention relates to crystalline vinflunine ditartrate in hydrated form. The number of water molecules is between 2 and 6 and preferentially between 3 and 6, for example it may be 2, 3, 4, 5 or 6.

The vinflunine ditartrate according to the invention advantageously has an infrared spectrum in KBr that shows an absorption peak at about 1730 cm⁻¹, several absorption bands between 1330 and 1420 cm⁻¹, an absorption band between 1275 and 1185 cm⁻¹, and two absorption bands between 1160 and 1030 cm⁻¹.

In one advantageous aspect, the X-ray diffraction spectrum of the vinflunine ditartrate that is the subject of the invention shows characteristic peaks, expressed in degrees 2θ, at about 5,641; 6,529; 7,991; 8,673; 9,245; 9,831; 11,369; 11,844; 12,273; 13,931; 14,334; 15,105; 15,805; 16,132; 16,833; 17,127; 17,461; 18,073; 18,711; 18,960; 19,835; 20,087; 20,629; 21,226; 21,414; 22,940; 23,662; 24,329; 25,064; 25,323; 25,959; 26,339; 27,600; 28,272; 29,006; 29,792; 30,525.

Vinflunine ditartrate of amorphous structure was able to be crystallized in a hydrated form in a solvent containing variable proportions of water. The solvent used is chosen from common water-miscible solvents, mainly alcohols. High temperatures will be avoided during the crystallization, on account of the fragility of the molecule.

The invention thus also relates to the process for preparing crystalline vinflunine ditartrate, characterized in that it comprises the following steps:
dissolving vinflunine ditartrate in an alcohol/water mixture,
slowly evaporating the solvent mixture at room temperature, in the open air or under vacuum,
filtering and recovering the crystals formed,
rinsing, and drying the crystals under vacuum.

The vinflunine ditartrate used for the implementation of the present invention is obtained according to the process described in patent application WO95/03312.

Preferably, the alcohol used is chosen from ethanol, 1-propanol and 2-propanol.

As indicated above, the dissolution temperature should be controlled so as to avoid any degradation of the molecule. Thus, a temperature below 70° C. and more particularly a temperature of 50° C. will advantageously be chosen.

The solvent used to dissolve the amorphous vinflunine ditartrate powder is water-miscible and chosen from alcohols. Advantageously, the alcohol/water ratio ranges between 75/25 and 100/0, and is preferably 80/20 by volume.

The amount of solvent will need to be adjusted by a person skilled in the art, and will preferably be between 1 and 20 parts by volume (ml) relative to the mass (g) of vinflunine ditartrate.

The crystals obtained are rinsed with a solvent that does not entrain any redissolution of the product, and will be performed, for example, using certain ether solvents, for example ethyl ether, isopropyl ether or methyl tert-butyl ether, and more particularly isopropyl ether.

The crystalline state of vinflunine ditartrate is demonstrated by means of techniques known to those skilled in the art, for instance X-ray powder diffraction or infrared spectrometry, and may be checked by simple microscopy.

On account of the therapeutic value, already demonstrated, of vinflunine and its derivatives, in particular salts, a subject of the present invention is also a medicament comprising the vinflunine ditartrate according to the invention. In one particular aspect, the invention relates to the use of crystalline vinflunine ditartrate for the preparation of a medicament intended to be used for treating cancer pathology. Mention may be made especially, in a non-limiting manner, of breast cancer, bladder cancer, non-small cell lung cancer and prostate cancer.

A subject of the invention is also a pharmaceutical composition, characterized in that it contains an effective amount of vinflunine ditartrate according to the invention, in a physiologically acceptable medium.

Among the pharmaceutical compositions that may be mentioned more particularly are those suitable for oral, parenteral, intravenous or subcutaneous administration, and more particularly suitable for oral administration, in the form of tablets, wafer capsules or gel capsules.

The dosage varies according to the sex, age and weight of the patient, and the route of administration.

The examples that follow illustrate the invention without limiting its scope.

### KEY TO THE FIGURES

FIG. 1: Observation by optical microscopy, in visible light, of crystalline vinflunine ditartrate, and of amorphous vinflunine ditartrate powder.

FIG. 2: Infrared spectra of crystalline vinflunine ditartrate and of the corresponding amorphous product. Percentage of transmission as a function of the wavenumber.

FIG. 3: Comparison of the infrared spectra of crystalline vinflunine ditartrate and of the corresponding amorphous product in the region 2000 cm⁻¹-800 cm⁻¹. Percentage of transmission as a function of the wave number.

FIG. 4: ¹H NMR spectrum of crystalline vinflunine ditartrate and of the corresponding amorphous product. Shifts in ppm.

FIG. 5: X-ray diffractogram of crystalline vinflunine ditartrate (dashed line) and of the corresponding amorphous product (solid line).

FIG. 6: List of the X-ray diffraction lines for crystalline vinflunine ditartrate.

### A. Crystallization of Vinflunine Ditartrate

### EXAMPLE 1

A sample of 7.5 g of vinflunine ditartrate is dissolved at 50° C. in 60 ml of 2-propanol containing 20% water. The solution is poured into a crystallizing basin, which is left in the open air at room temperature for several days. The crystals formed are then collected by filtration if the evaporation of the solvent is incomplete, or by simple scraping of the walls if all the solvent has evaporated off. The crystals obtained are rinsed with isopropyl ether and then dried under vacuum.

Elemental analysis:

| | |
|---|---|
| C₅₃H₆₆N₄F₂O₂₀: | 1117.12 |
| Theory %: | C 56.98, H 5.95, N 5.02 |
| Found %: | C 52.51, H 5.78, N 4.69 |
| Corrected (H₂O 6.59%): | C 56.21, H 5.40, N 5.03 |

### EXAMPLE 2

A sample of 7.5 g of vinflunine ditartrate is dissolved at 50° C. in 60 ml of 2-propanol containing 20% water. The solution is poured into a crystallizing basin that is placed in a vacuum chamber at 25° C. for several days. The crystals formed are then collected by filtration if the evaporation of the solvent is incomplete, or by simple scraping of the walls if all of the solvent has evaporated off. The crystals obtained are rinsed with isopropyl ether and then dried under vacuum.

Elemental analysis:

| | |
|---|---|
| C₅₃H₆₆N₄F₂O₂₀: | 1117.12 |
| Theory %: | C 56.98, H 5.95, N 5.02 |
| Found %: | C 52.47, H 5.91, N 4.61 |
| Corrected (H₂O 6.6%): | C 56.17, H 5.53, N 4.94 |

### EXAMPLE 3

A sample of 200 mg of vinflunine ditartrate is dissolved at 50° C. in 10 ml of 1-propanol containing 20% water. The solution is poured into a crystallizing basin that is left in the open air at room temperature for several days. The crystals formed are then collected by simple scraping of the walls when all of the solvent has evaporated off. The crystals obtained are rinsed with isopropyl ether and then dried under vacuum.

Elemental analysis:

| | |
|---|---|
| C₅₃H₆₆N₄F₂O₂₀: | 1117.12 |
| Theory %: | C 56.98, H 5.95, N 5.02 |
| Found %: | C 53.64, H 6.36, N 4.75 |
| Corrected (H₂O 6.46%): | C 57.34, H 6.03, N 5.08 |

### B. Characterization of the Crystalline Vinflunine Ditartrate According to the Invention

### Optical Microscopy in Visible Light

The vinflunine ditartrate powder is examined in visible light using a Continuum microscope equipped with the following accessories:
trinocular with 10× eyepieces
STI high-resolution colour camera, version NTSC.
4 MB GXT video card
mView software version 2.6a
visible polariser/analyser

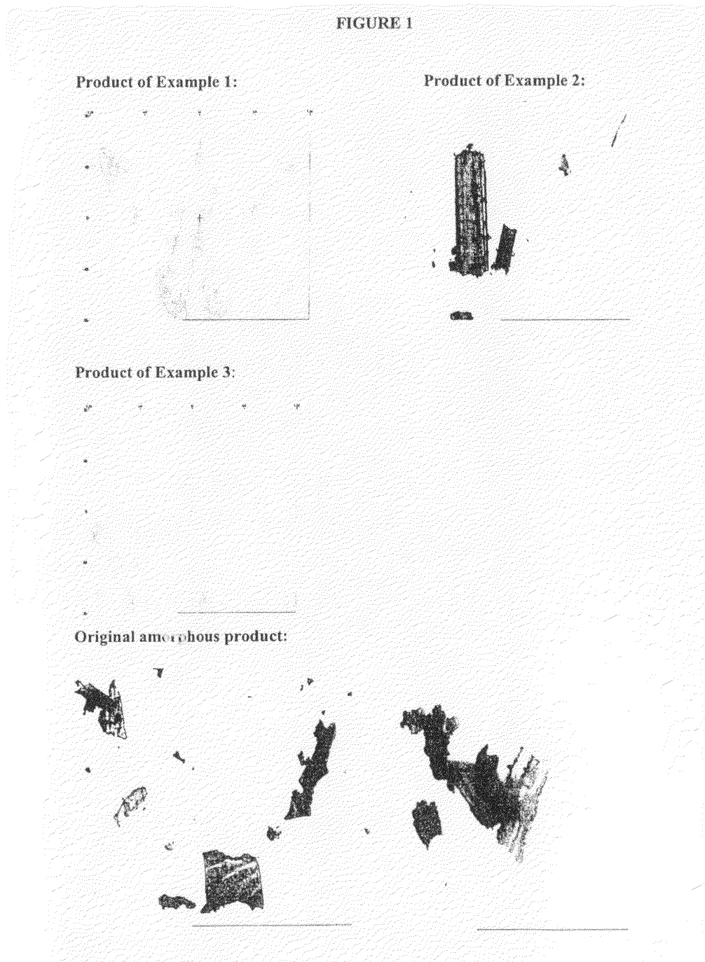

The results of the observations are given in FIG. 1: an organized crystalline system is observed for each of the samples obtained in Examples 1, 2 and 3, but not in the case of the sample of the amorphous product.

### Infrared Spectroscopy

The infrared spectrum is recorded on a Nexus model 670 FT-IR spectrometer coupled to a Continuμm microscope (ThermoElectron).

A sample of about 1 mg of vinflunine ditartrate is placed on a potassium bromide disc. The infrared spectrum is recorded on a crystal of this powder using the following instrument parameters:
Continuμm microscope:
Transmission mode
MCT-A detector
Reflachromat 32× “infinity corrected” objective and condenser with variable compensation
Optical block:
Nexus 670 FT-IR spectrometer accreditation COFRAC (No. 1-1009)
Vectra interferometer
Ever Glo source, resolution 0.5 cm⁻¹
KBr separator (7400-350 cm⁻¹)
Omnic® software version 6.2
Number of sweeps: 256
Resolution 8
Happ-Genzel apodization function
Phase correction: Mertz

Results:

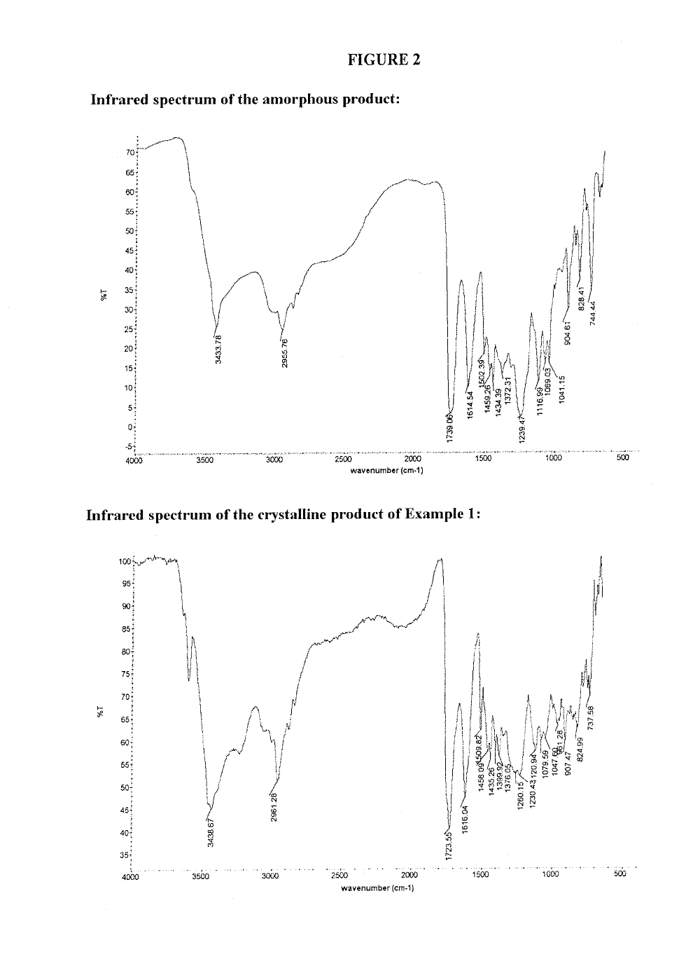

The resulting spectra obtained for the amorphous product and for the crystalline product according to Example 1 are given in FIG. 2.

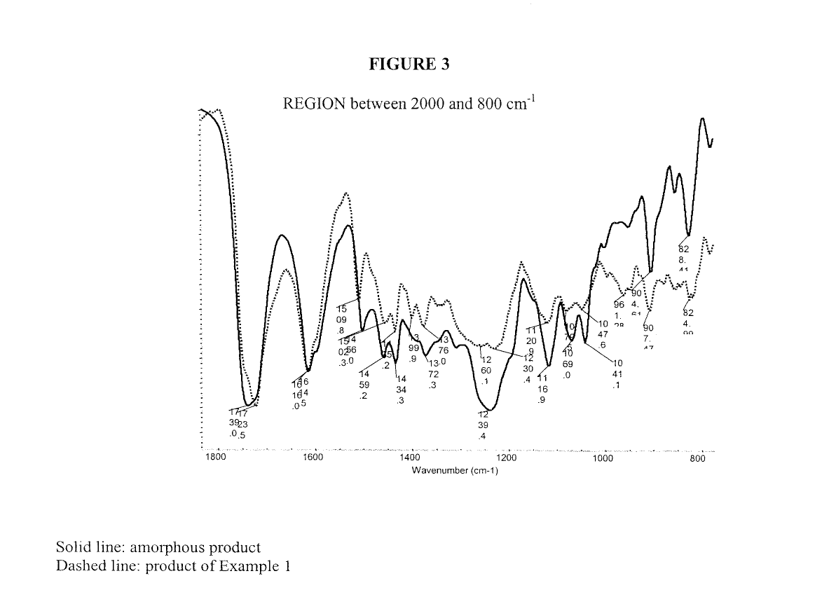

A comparative analysis between these two spectra for the regions between 2000 cm⁻¹ and 800 cm⁻¹ is given in FIG. 3.

The strong absorption band observed for the two products at about 1730 cm⁻¹ is characteristic of the stretching vibration of the carbonyl groups C=O.

The broad absorption band between 1275 and 1185 cm⁻¹ is derived from the asymmetrical stretching vibrations of the ester groups C—O—C. The absorption bands between 1160 and 1030 cm⁻¹ are due to the symmetrical stretching vibrations of the ester groups C—O—C. These relatively strong bands are representative of the various aliphatic esters present in the vinflunine molecule.

The bending vibrations in the plane of the tertiary alcohol function O—H give rise to absorption bands between 1420 and 1330 cm⁻¹.

The shape and vibration frequency of these absorption bands are significantly different between the two polymorphic species.

### Nuclear Magnetic Resonance

The ¹H NMR spectrum is recorded at a nominal frequency of 400 MHz on a Bruker Avance DPX 400 spectrometer equipped with a broad-band inverse probe and a z gradient accessory. Before recording the NMR spectrum, the product is predissolved in deuterated methanol (Eurisotop, reference D 324-B, batch A-3561) at a concentration in the region of 0.4% (w/v). The chemical shifts are expressed in ppm relative to TMS (tetramethylsilane) used as internal standard. The coupling constants are expressed in Hertz.

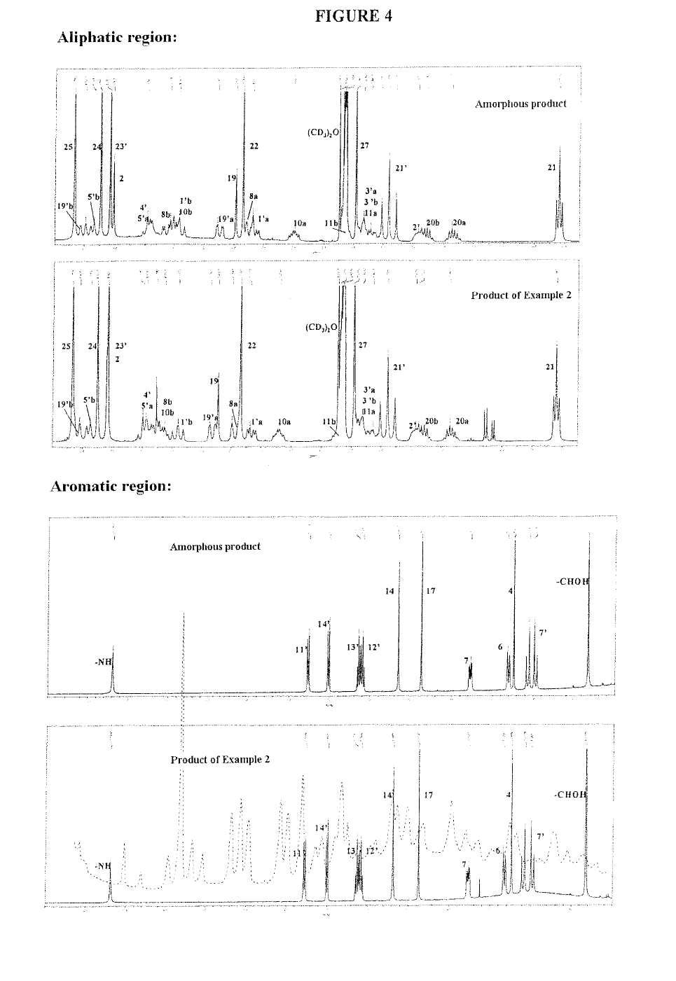

FIG. 4 collates the spectra obtained for the amorphous product and for the product of Example 2, comparatively:

the two spectra are comparable and in accordance with the chemical structure of vinflunine ditartrate. The differences observed between the two NMR spectra are mainly due to the concentration differences between the two samples; the crystalline batch also contains crystallization solvents.

Nuclear magnetic resonance is used firstly to confirm the structural integrity of the vinflunine ditartrate molecule after the crystallization test, and secondly to determine the tartaric acid/vinflunine mole ratio. This ratio is 2/1 for the two polymorphic species amorphous and crystalline); this result being confirmed by elemental analysis.

### Powder X-Ray Diffraction

The samples were analysed on a D8 Advance Bruker AXS diffractometer equipped with a copper anticathode (λ=1.54060 Å) operating with a voltage of 40 kV and a current of 40 mA, a variable primary slit block and a Vantec detector.

The analyses were performed between 3 and 35° 2θ with an interval of 0.030°2θ and a counting time of 40 seconds. Given the cytotoxic nature of the molecule, the samples were held in a confined environment using a 25 mm sample holder supported by a transparent hermetic dome (A100B33 Bruker AXS). The samples were then analysed by HPLC to ensure that the X-rays did not degrade the samples.

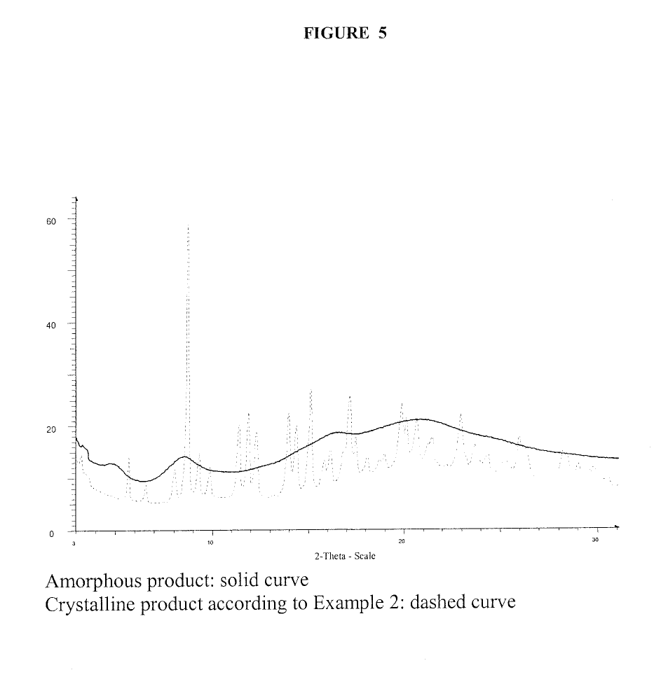

The diffractograms of FIG. 5 show that the product of Example 2 is crystalline, whereas the original product is amorphous.

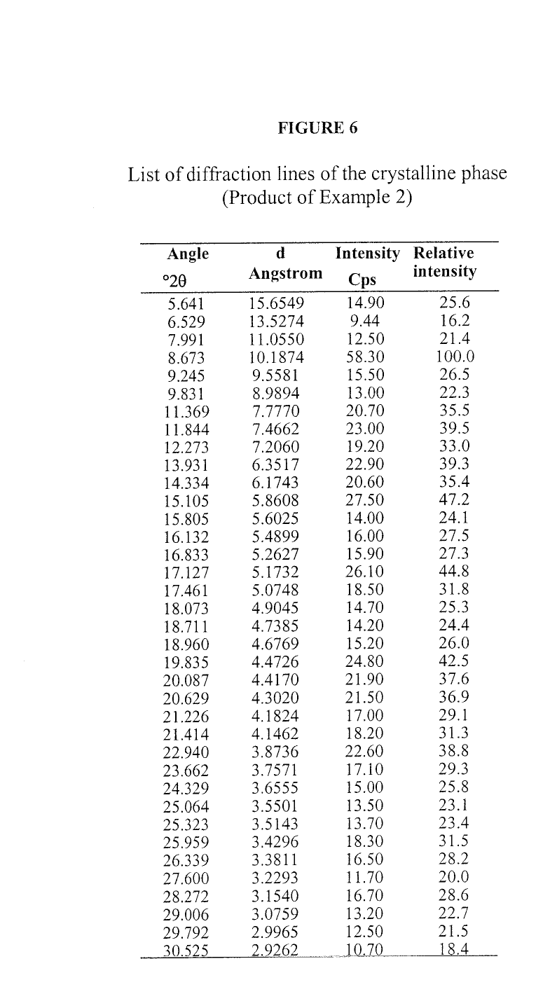

The crystalline state is characterized by the list of diffraction lines presented in the table in FIG. 6.

The HPLC analysis does not show any significant degradation of the products after exposure to X-rays.

1. Crystalline vinflunine ditartrate.

2. Vinflunine ditartrate according to claim 1, characterized in that it is in hydrated form.

3 Vinflunine ditartrate according to claim 2, characterized in that the number of water molecules is between 2 and 6.

4. Vinflunine ditartrate according to claim 1, the infrared spectrum of which in KBr shows an absorption peak at about 1730 cm⁻¹, several absorption bands between 1330 and 1420 cm⁻¹, an absorption band between 1275 and 1185 cm and two absorption bands between 1160 and 1030 cm⁻¹.

5. Crystalline form of vinflunine ditartrate according to claim 1, having an X-ray diffraction spectrum showing characteristic peaks, expressed in degrees 2θ, at about 5,641; 6,529; 7,991; 8,673; 9,245; 9,831; 11,369; 11,844; 12,273; 13,931; 14,334; 15,105; 15,805; 16,132; 16,833; 17,127; 17,461; 18,073; 18,711; 18,960; 19,835; 20,087; 20,629; 21,226; 21,414; 22,940; 23,662; 24,329; 25,064; 25,323; 25,959; 26,339; 27,600; 28,272; 29,006; 29,792; 30,525.

6. Process for preparing crystalline vinflunine ditartrate according to claim 1, comprising the steps of:
dissolving vinflunine ditaitrate in an alcohol/water mixture,
slowly evaporating the solvent mixture at room temperature, in the open air or under vacuum,
filtering and recovering the crystals formed.
rinsing, and drying the crystals under vacuum.

7. Preparation process according to claim 6, characterized in that the alcohol used is chosen from ethanol, 1-propanol and 2-propanol.

8. Preparation process according to claim 6, characterized in that the dissolution is performed by heating to a temperature below 70° C. and preferentially to 50° C.

9. Preparation process according to claim 6, characterized in that the alcohol/water ratio ranges between 75/25 and 100/0 by volume.

10. Preparation process according to claim 6, characterized in that the proportion of solvent is between 1 and 20 parts by volume expressed in millilitres relative to the mass in grams of vinflunine ditartrate.

11. Preparation process according to claim 6, characterized in that the rinsing is performed using an ether chosen from ethyl ether, isopropyl ether and methyl tert-butyl ether.

12. Vinflunine ditartrate according to claim 1, as a medicament.

13. Pharmaceutical composition, characterized in that it comprises an effective amount of vinflunine ditartrate according to claim 1 in a physiologically acceptable medium.

14. Use of vinflunine ditartrate according to claim 1 for the preparation of a-medicament intended to be used for treating cancer pathlology.
